(19) United States
(12) Patent Application Publication
(10) Pub. No.: US 2003/0139378 A1
(43) Pub. Date: Jul. 24, 2003

(54) Liquid bisphosphonate formulations for bone disorders

(76) Inventors: Anastasia G. Daifotis, Westfield, NJ (US); Albert T. Leung, Ringoes, NJ (US); Andrew E. Denker, Hoboken, NJ (US); Paul A. Bergquist, Collegeville, PA (US)

Correspondence Address: MERCK AND CO INC, P O BOX 2000, RAHWAY, NJ, 070650907

(21) Appl. No.: 10/305,868

(22) Filed: Nov. 26, 2002

Related U.S. Application Data
(60) Provisional application No. 60/340,965, filed on Dec. 13, 2001.

(51) Int. Cl. A61K 31/675; A61K 31/663

(52) U.S. Cl. 514/89; 514/102

(57) ABSTRACT

The present invention relates to high dose oral liquid formulations of bisphosphonate and their methods of use to treat/prevent diseases related to bone remodeling or bone disorders, such as for example, Paget's disease, osteoporosis, metastatic bone disease, hypercalcemia of malignancy, periprosthetic osteolysis, periodontal disease, arthritic conditions, and the like, while minimizing the potential for esophageal irritation and other adverse gastrointestinal effects. These methods comprise orally administering to a mammal in need thereof a pharmaceutically effective amount of the liquid pharmaceutical composition of at least one bisphosphonate, or a pharmaceutically acceptable salt thereof, as a unit dosage according to a continuous schedule having a once-weekly, twice-weekly, biweekly, twice-monthly, or monthly dosing interval. The present invention also relates to liquid pharmaceutical compositions of the bisphosphonate for carrying out these methods.

### FIELD OF THE INVENTION

The present invention relates to high dose oral liquid formulations of bisphosphonate and their methods of use to treat/prevent diseases related to bone remodeling or bone disorders, such as for example, Paget's disease, osteoporosis, metastatic bone disease, hypercalcemia of malignancy, periprosthetic osteolysis, periodontal disease, arthritic conditions, and the like, while minimizing the potential for esophageal irritation and other adverse gastrointestinal effects. These methods comprise orally administering to a mammal in need thereof a pharmaceutically effective amount of the liquid pharmaceutical composition of at least one bisphosphonate, or a pharmaceutically acceptable salt thereof, as a unit dosage according to a continuous schedule having a once-weekly, twice-weekly, biweekly, twice-monthly, or monthly dosing interval. The present invention also relates to liquid pharmaceutical compositions of the bisphosphonate for carrying out these methods.

### BACKGROUND OF THE INVENTION

Paget's disease, osteoporosis, metastatic bone disease, hypercalcemia of malignancy, periprosthetic osteolysis, periodontal disease, and arthritic conditions constitute bone disorders in humans and other mammals that are characterized by abnormal bone remodeling. Because these bone disorders are chronic conditions, appropriate therapy generally requires long-term treatment.

Paget's disease of bone is a chronic progressive skeletal bone disorder in which giant multinucleated cells called osteoclasts dramatically activate bone resorption which is followed by excessive bone formation. This accelerated bone resorption/bone formation cycle results in increased and disordered bone remodeling, bone hypertrophy, abnormal and weakened bone structure, and abnormal mechanical properties. Clinical consequences include severe bone pain, bone enlargement, bone deformity, and increased blood flow. Other complications can include bone pain due to deformity of bones adjacent to joints, entrapment of nerves, especially if the spine or the base of the skull is affected, and fractures [see W. D. Fraser et al., “Paget's disease of bone,” Current Opinion in Rheumatology, 9: 347-354 (1997)].

Osteoporosis is a systemic skeletal disease characterized by a low bone mass and microarchitectural deterioration of bone tissue, with a consequent increase in bone fragility and susceptibility to fracture. Osteoporosis, can affect men, patients treated with glucocorticoid and, in its most frequent manifestation, postmenopausal women [see John A. Kanis, Osteoporosis 81-111 (Blackwell Science, Inc. 1994)].

Metastatic bone disease is a condition in which tumors, of various origins, in particular breast, lung, prostate, and myeloma, migrate (metastasize) to bone and induce bone resorption and subsequent bone destruction leading to pain, fractures, hypercalcemia, and nerve compression syndromes. The metastasized tumor cells locally resorb bone mainly through the production or the induction of bone resorbing cytokines. Breast cancer is particularly associated with metastatic bone disease in that the majority of patients with advanced breast cancer manifest osteolytic bone metastases [see R. E. Coleman, “How can we improve the treatment of bone metastases further?” Current Op. Onc., 10 (Supp. 1): S7-13.] A growing body of evidence shows that bisphosphonates have a direct effect on tumor cells. Animal studies have shown that bisphosphonates reduce osteolysis, skeletal-related events, and decrease tumor burden, suppression of bone resorption in even other wise normal bone resoption, and makes “the skeleton a less hospitable site for tumor cells to grow and behave agressively.” See “Bisphosphonates and Tumor Burden,” J Clin Oncol August 2002; 20(15):3191-3992. Thus, bisphosphonates can be useful in the prevention and treatment of metastatic bone disease. See, Green, J. R., “Bisphosphonates in cancer therapy,” Curr Opin Oncol November 2002; 14(6):609-615; Saad, F., et al., “A Randomized, Placebo-Controlled Trial of Zoledronic Acid in Patients with Hormone-Refractory Metastatic Prostate Carcinoma,” JNIC October 2002; 94(19): 1458-1468.

Tumors of various origins outside the skeleton can also induce bone resorption through the systemic secretion of osteolytic factors, mostly parathyroid hormone-related protein. Bone resorption can lead to generalized bone destruction which can be accompanied by hypercalcemia, referred to as humoral hypercalcemia of malignancy. The hypercalcemia in tumor bone disease is caused essentially by increase osteolysis, as well as by increased renal reabsorption of calcium and dehydration. The hypercalcemia, which affects mainly the nervous, gastrointestinal, cardiovascular, and renal systems, can be life-threatening. Hypercalcemia of malignancy is particularly associated with carcinoma of the lung, breast, skin, head and neck, kidney, bladder, and ovary, as well as myeloma [see Stewart, Andrew F. and Broadus, Arthur E, “Malignancy-Associated Hypercalcemia,” In: DeGroot, Leslie J. and Jameson, J. Larry, eds. Endocrinology. 4th ed., Philadelphia: W. B. Saunders Company; 2001: 1093-1100].

Periprosthetic osteolysis is a bone disease in which debris particles produced by the wear of the articular surfaces of prosthetic joint induce an immune response and activation of osteoclasts, resulting in bone resorption and destruction in the area around the prosthesis. Periprosthetic osteolysis eventually results in loosening of the prosthetic joint, pain, and fracture [see Harris W H, “The problem is osteolysis,” Clin. Orthop. 1995;(311):46-53].

Periodontal diseases inflame and destroy the structures surrounding and supporting the teeth, primarily the gums, bone, and the outer layer of the tooth root. Periodontal diseases are caused mainly by accumulation of bacteria and can be affected by some general body conditions, such as diabetes mellitus, poor nutrition, leukemia, and AIDS as well as by smoking. Studies have shown that bisphosphonates are useful in the treatment and diagnosis of periodontal diseases. See, Rocha, M. et al., “Clinical and radiological improvement of periodontal disease in patients with type 2 diabetes mellitus treated with alendronate: a randomized, placebo-controlled trial,” J Periodontol February 2001; 72(2):204-9, and Tenenbaum, H. C., et al., “Bisphosphonates and periodontics: potential applications for regulation of bone mass in the periodontium and other therapeutic/diagnostic uses,” J Periodontol July 2002;73(7):813-22.

“Arthritic condition” or “arthritic conditions” refers to a disease wherein inflammatory lesions are confined to the joints or any inflammatory conditions of the joints, most notably osteoarthritis and rheumatoid arthritis (Academic Press Dictionary of Science Technology; Academic Press; 1st edition, Jan. 15, 1992). The compositions of the present invention are also useful, alone or in combination, to treat or prevent arthritic conditions, such as Behcet's disease; bursitis and tendinitis; CPPD deposition disease; carpal tunnel syndrome; Ehlers-Danlos syndrome; fibromyalgia; gout; infectious arthritis; inflammatory bowel disease; juvenile arthritis; lupus erythematosus; lyme disease; marfan syndrome; myositis; osteoarthritis; osteogenesis imperfecta; osteonecrosis; polyarteritis; polymyalgia rheumatica; psoriatic arthritis; Raynaud's phenomenon; reflex sympathetic dystrophy syndrome; Reiter's syndrome; rheumatoid arthritis; scleroderma; and Sjogren's syndrome. An embodiment of the invention encompasses the treatment or prevention of an arthritic condition which comprises administering a therapeutically effective amount of a composition of the present invention. A subembodiment is the treatment or prevention of osteoarthritis which comprises administering a therapeutically effective amount of a composition of the present invention. See: Cutolo M, Seriolo B, Villaggio B, Pizzorni C, Craviotto C, Sulli A. Ann. N.Y. Acad. Sci. June 2002;966:131-42; Cutolo, M. Rheum Dis Clin North Am November 2000;26(4):881-95; Bijlsma J W, Van den Brink H R. Am J Reprod Immunol October-December 1992;28(3-4):231-4; Jansson L, Holmdahl R.; Arthritis Rheum September 2001;44(9):2168-75; and Purdie D W. Br Med Bull 2000;56(3):809-23. Also, see Merck Manual, 17th edition, pp. 449-451.

Osteoarthritis (OA) is a connective tissue disease, with pathology arising from mechanical insult-induced articular cartilage degeneration, a limited synovial inflammatory response, and subchondral bone remodeling. The net outcome of these activities is joint deformity secondary to erosion of the articular surface, peri-articular endochondral ossification/osteophytosis, and subchondral bony sclerosis and cyst formation [R Oettmeier, K. Abendroth “Osteoarthritis and bone: osteologic types of osteoarthritis of the hip”, Skeletal Radiol 18: 165-174 (1989)]. Although, OA is considered primarily a cartilaginous disorder, it is accompanied by well-defined changes in trabecular bone of the joint, and it is possible that the subchondral bone changes can potentially contribute to the initiation and progression of OA [K Senior. “Osteoarthritis research: on the verge of a revolution? Lancet 355: 208 (2000)].

An effective clinical approach to the control of abnormal bone remodeling in diseases, such as Paget's disease, osteoporosis, metastatic bone disease, hypercalcemia of malignancy, periprosthetic osteolysis, and arthritic conditions, is treatment with an antiresorptive agent, for example, a bisphosphonate, including clodronate, pamidronate, alendronate, tiludronate, risedronate, zoledronate, and ibandronate [see H. Fleisch, “Bisphosphonates: mechanisms of action,” Exp. Opin. Ther. Patents, 11: 1371-1381 (2001) and T. J. Schnitzer et al., “Update on alendronate for osteoporosis: once-weekly dosing,” Exp. Opin. Pharmacother., 2: 1461-1472 (2001)]. Treatment of Paget's disease with bisphosphonates has been observed to afford not only reduction but also suppression of disease activity. Etidronate was the first bisphosphonate to be used in the clinic for Paget's disease; however, the dose and duration of therapy were limited by its tendency to impair bone mineralization [see E. Siris et al., “Comparative Study of Alendronate Versus Etidronate for the Treatment of Paget's Disease of Bone,” J. Clin. Endocrinol. Metab., 81: 961-967 (1996)]. Intravenous pamidronate has also proven effective in patients unresponsive to treatment with etidronate. Alendronate sodium is a potent orally administered nitrogen-containing bisphosphonate which has been approved for treatment of Paget's disease in several countries, including the United States and Canada. The dosing regimen of alendronate in the treatment of Paget's disease is typically 40 mg per day for 6 months. This treatment modality has been characterized by marked reductions in serum alkaline phosphatase in most patients, normalization of alkaline phosphatase in the majority of patients, and improvement in osteolytic lesions [see E. S. Siris, “A Potent New Bisphosphonate for Paget's Disease of Bone,” Am. J. Med., 101: 339-340 (1996); S. A. Khan et al., “Alendronate in the Treatment of Paget's Disease of Bone,” Bone, 20: 263-271 (1997); and A. Lombardi, “Treatment of Paget's Disease of Bone with Alendronate,” Bone, 24: 59S-61S (1999)]. Risedronate has also been approved in the U.S. for the treatment of Paget's disease. The recommended treatment regimen for risedronate is 30 mg orally once daily for 2 months. Retreatment can be considered if relapse occurs.

The use of bisphosphonates in the treatment of diseases characterized by abnormal bone remodeling is documented in H. Fleisch, “Bisphosphonates: Mechanisms of Action,” Exp. Opin. Ther. Patents, 11: 1371-1381 (2001) and H. Fleisch, “Bisphosphonates in Bone Disease,” 4^{th} edition, Academic Press, New York, 2000.

Despite their proven therapeutic benefits in patients with disorders associated with abnormal bone remodeling, bisphosphonates are poorly absorbed from the gastrointestinal tract, which is further compromised by the presence of food and beverages other than water which can bind the bisphosphonates and further diminish their bioavailability. Intravenous administration has been used to overcome this shortcoming in bioavailability. For example, intravenous pamidronate is approved in the U.S. for use in Paget's disease. However, intravenous administration is costly and inconvenient, particularly when the patient must be given an intravenous infusion lasting several hours on repeated occasions.

Therefore, for the treatment of bone disorders, such as Paget's disease, osteoporosis, metastatic bone disease, hypercalcemia of malignancy, periprosthetic osteolysis, periodontal disease, arthritic conditions and the like, oral administration remains one administration route for bisphosphonates, such as alendronate and risedronate. To facilitate adequate absorption from the gastrointestinal tract, it is generally recommended that oral bisphosphonates, such as alendronate and risedronate, be taken in the fasting state, 30 minutes before the first food, beverage, or medication of the day. This limitation on the use of oral bisphosphonates is a source of considerable inconvenience to patients in need of therapy and can result in decreased compliance. Furthermore, esophageal irritation has been observed with oral bisphosphonate use, albeit infrequently [for alendronate use, see P. C. De Groen, “Esophagitis associated with the use of alendronate,” New Engl. J. Med., 335: 1016-1021 (1996)].

The mechanism of the esophagitis involves a local effect on the esophagus either due to the bisphosphonate tablets adhering to the esophagus or refluxing of gastric content containing dissolved bisphosphonate. To minimize the potential for esophageal irritation, a bisphosphonate, such as alendronate and risedronate, should be taken in the upright position, with a full glass of water, and the patient should not lie down until after eating. Gastrointestinal intolerance can lead to discontinuance in the use of the drug for treatment of the various bone disorders associated with abnormal bone remodeling.

The degree of adverse gastrointestinal effects of alendronate has been shown to increase with increasing daily dose [see C. H. Chesnut et al., “Alendronate Treatment of the Postmenopausal Osteoporotic Woman: Effect of Multiple Dosages on Bone Mass and Bone Remodeling,” Am. J. Med., 99: 144-152 (1995) and C. H. Chesnut et al., “Short-term effect of alendronate on bone mass and bone remodeling in postmenopausal women,” Osteoporosis Int. (Suppl. 3), S17-19 (1993)]. In clinical studies, there was an apparent increased incidence of upper gastrointestinal adverse experiences in patients taking 40 mg/day of alendronate as compared with the approved 10 mg per day dose for the continuous treatment of osteoporosis. The dosing regimen of alendronate for Paget's disease is 40 mg/day for 6 months, to be distinguished from the approved 10 mg/day dose for the continuous treatment of established osteoporosis in postmenopausal women and 5 mg/day dose for the prevention of osteoporosis in postmenopausal women and the treatment of glucocorticoid-induced osteoporosis in men and women.

Oral bisphosphonate therapies generally fall into three categories: (1) those therapies utilizing continuous daily treatment, (2) those therapies utilizing a cyclic regimen of treatment and rest periods, and (3) those therapies utilizing a relatively high solid unit dosage at a low relative dosing frequency.

The continuous daily treatment regimens normally involve the chronic administration of relatively low doses of the bisphosphonate compound, with the objective of delivering the desired cumulative therapeutic dose over the course of the treatment period. However, continuous daily dosing has the potential disadvantage of causing adverse gastrointestinal effects due to the repetitive, continuous, and additive irritation to the gastrointestinal tract. Moreover, because bisphosphonates should be taken on an empty stomach followed by fasting and maintenance of an upright posture for at least 30 minutes, many patients find daily dosing to be burdensome. These factors can therefore interfere with patient compliance, and in some cases even result in cessation of treatment.

Generally, cyclic regimens are characterized as being intermittent, as opposed to continuous treatment regimens, and have both treatment periods during which the bisphosphonate is administered and nontreatment periods to permit the systemic level of the bisphosphonate to return toward baseline. However, the cyclic regimens, relative to continuous dosing, appear to result in a decreased therapeutic antiresorptive efficacy. Furthermore, these cyclic regimens do not eliminate or minimize adverse gastrointestinal effects, because such regimens typically utilize periods of multiple daily dosing. Also, the cyclic regimens are cumbersome to administer and have the disadvantage of low patient compliance, and consequently compromised therapeutic efficacy.

However, regimens wherein the bisphosphonate, such as alendronate and risedronate, is administered at a relatively high unit dose but at a low relative dosing frequency cause less adverse gastrointestinal effects, particularly esophageal effects, compared to the administration of a low relative dose at a high relative dosing frequency. When compared with 10 mg of alendronate sodium daily, the administration of 70 mg of alendronate sodium once-weekly for the treatment of postmenopausal osteoporosis has been observed to cause fewer incidences of adverse gastrointestinal events and the resulting adverse gastrointestinal events are no different than what is observed with placebo [T. Schnitzer et al., “Therapeutic equivalence of alendronate 70 mg once-weekly and alendronate 10 mg daily in the treatment of osteoporosis,” Aging Clin. Exp. Res., 12: 1-12 (2000)].

### SUMMARY OF THE INVENTION

The present invention relates to high dose oral liquid formulations of bisphosphonate and their methods of use to treat/prevent diseases related to bone remodeling or bone disorders, such as for example, Paget's disease, osteoporosis, metastatic bone disease, hypercalcemia of malignancy, periprosthetic osteolysis, periodontal disease, arthritic conditions, and the like, while minimizing the potential for esophageal irritation and other adverse gastrointestinal effects. These methods comprise orally administering to a mammal in need thereof a pharmaceutically effective amount of the liquid pharmaceutical composition of at least one bisphosphonate, or a pharmaceutically acceptable salt thereof, as a unit dosage according to a continuous schedule having a once-weekly, twice-weekly, biweekly, twice-monthly, or monthly dosing interval. The present invention also relates to liquid pharmaceutical compositions of the bisphosphonate for carrying out these methods.

One embodiment of the invention relates to a method for treating or preventing a bone disorder in a mammal in need thereof comprising orally administering to said mammal a pharmaceutically effective amount of a liquid pharmaceutical composition of at least one bisphosphonate, or a pharmaceutically acceptable salt thereof, as a unit dosage which ranges from greater than about 140 mg to about 1120 mg, on a bisphosphonic acid active basis, according to a dosing schedule having a dosing interval selected from once-weekly dosing, twice-weekly dosing, biweekly dosing, twice-monthly dosing, and once-monthly dosing.

In other embodiments, the present invention is concerned with a liquid pharmaceutical composition comprising about greater than about 140 mg to about 560 mg, on an alendronic acid active basis, of at least one bisphosphonate and pharmaceutically acceptable salts thereof, or mixtures thereof, in an aqueous solution. In a class of these embodiments, the liquid pharmaceutical composition comprises about greater than about 140 mg to about 560 mg, on an alendronic acid active basis, of alendronate, a pharmaceutically acceptable salt thereof, or mixtures thereof, in about 30 mL to about 150 mL of a buffered aqueous solution. In a subclass of this class, the liquid pharmaceutical composition comprises about 75 mL of a buffered aqueous solution.

All percentages and ratios used herein, unless otherwise indicated, are indicated here for convenience by weight. The invention hereof can comprise, consist of, or consist essentially of the essential as well as optional ingredients, components, and methods described therein.

### DETAILED DESCRIPTION OF THE INVENTION

The present invention relates to liquid oral formulations of at least one bisphosphonate and their use to treating and/or preventing abnormal bone remodeling associated with the bone disorders of Paget's disease; osteoporosis, such as for example, osteoporosis in men, postmenopoausal women, and patients treated with glucocorticoid; metastatic bone disease, hypercalcemia of malignancy; periprosthetic osteolysis;periodontal disease; and arthritic conditions, including osteoarthritis and rheumatoid arthritis in a mammal in need thereof, while minimizing the occurrence of or potential for esophageal irritation and other adverse gastrointestinal effects. The methods of the present invention comprise orally administering to said mammal an oral buffered solution comprising a high dose of at least one, or a pharmaceutically acceptable salt thereof, as a unit dosage according to a continuous dosing schedule having a once-weekly, twice-weekly, biweekly, twice-monthly, or once-monthly interval. Typically, the continuous dosing schedule is maintained until the desired therapeutic effect is achieved for the mammal.

The present invention utilizes high unit dosages of bisphosphonate. The dosing schedule chosen and the properties of the formulations unexpectedly minimize the potential for esophageal irritation and other adverse gastrointestinal effects.

Besides not causing esophageal irritation associated with the solid dosage forms of bisphosphonates used for Paget's disease, osteoporosis, metastatic bone disease, hypercalcemia of malignancy, periprosthetic osteolysis, and arthritic conditions, the buffered liquid formulations of the present invention are also advantageous in that a bisphosphonate is often prescribed to elderly patients who experience difficulty in swallowing tablets or capsules, but can more readily swallow a non-irritating liquid formulation.

The methods of the present invention are generally administered to mammals with Paget's disease, osteoporosis, metastatic bone disease, hypercalcemia of malignancy, periprosthetic osteolysis, periodontal disease and arthritic conditions. In particular the mammals are human patients.

Definitions

The term “high dose” is defined as a unit dose that delivers greater than about 140 mg of at least one bisphosphonate or a pharmaceutically acceptable salt thereof, on an alendronic acid weight basis.

The term “minimize the occurrence of or potential for adverse upper gastrointestinal effects,” as used herein, means reducing, preventing, decreasing, or lessening the occurrence of or the potential for incurring unwanted side effects in the gastrointestinal tract, i.e. the esophagus and stomach, and upper intestines. Nonlimiting adverse upper gastrointestinal effects include, but are not limited to, gastroesophageal reflux disease (GERD), esophagitis, dyspepsia, ulcers, esophageal irritation, esophageal perforation, gastritis, and abdominal pain.

The term “abnormal bone remodeling inhibiting,” as used herein, means treating or preventing abnormal bone remodeling by inhibiting excessive osteoclastic bone resorption that characterizes Paget's disease, osteoporosis, metastatic bone disease, hypercalcemia of malignancy, periodontal disease, periprosthetic osteolysis, and arthritic conditions, resulting in bone loss and, in the case of Paget's disease, in the production of abnormal bone as a result of a compensatory increase in new bone formation.

The term “abnormal bone resorption,” as used herein, means a degree of bone resorption that exceeds the degree of bone formation, either locally, or in the skeleton as a whole. Alternatively, “abnormal bone resorption” can be associated with the formation of bone having an abnormal structure.

“Diseases related to bone remodeling” includes, but is not limited to, diseases characterized by abnormal bone resorption as defined above and conditions that would be inhibited by suppressing otherwise normal bone resorption, such as metastatic bone disease and the like.

The term “bone resorption inhibiting,” as used herein, means treating or preventing bone resorption by the direct or indirect alteration of osteoclast formation or activity. Inhibition of bone resorption refers to treatment or prevention of bone loss, especially the inhibition of removal of existing bone either from the mineral phase and/or the organic matrix phase, through direct or indirect alteration of osteoclast formation or activity.

The term “continuous schedule” or “continuous dosing schedule,” as used herein, means that the dosing regimen is repeated until the desired therapeutic effect is achieved. The continuous schedule or continuous dosing schedule is distinguished from cyclical or intermittent administration.

The term “until the desired therapeutic effect is achieved,” as used herein, means that the bisphosphonate is continuously administered, according to the dosing schedule chosen, up to the time that the clinical or medical effect sought for the disease or condition is observed by the clinician or researcher. For methods of treatment of the present invention, the bisphosphonate is continuously administered until the desired change in bone mass or structure is observed. In such instances, achieving an increase in bone mass or a replacement of abnormal bone structure with more normal bone structure is the desired objective. For methods of prevention of the present invention, the bisphosphonate is continuously administered for as long as necessary to prevent the undesired condition. In such instances, maintenance of normal bone structure is often the objective. Nonlimiting examples of administration periods can range from about 2 weeks to the remaining lifespan of the mammal. For humans, administration periods can range from about 2 weeks to the remaining lifespan of the human, such as for example, from about 2 weeks to about 20 years, further from about 1 month to about 20 years, further still from about 6 months to about 10 years, and even further from about 1 year to about 10 years.

Bisphosphonates

The bisphosphonates which can also be employed in the methods and compositions of the present invention include:

(a) Alendronate, also known as Alendronic acid, 4-amino-1-hydroxybutylidene-,1-bisphosphonic acid, alendronate sodium, alendronate monosodium trihydrate or 4-amino-1-hydroxybutylidene-1,1-bisphosphonic acid monosodium trihydrate. Alendronate is described in U.S. Pat. Nos. 4,922,007, to Kieczykowski et al., issued May 1, 1990; 5,019,651, to Kieczykowski et al., issued May 28, 1991; 5,510,517, to Dauer et al., issued Apr. 23, 1996; 5,648,491, to Dauer et al., issued Jul. 15, 1997, all of which are incorporated by reference herein in their entirety;

(b) [(cycloheptylamino)-methylene]-bis-phosphonate (incadronate), which is described in U.S. Pat. No. 4,970,335, to Isomura et al., issued Nov. 13, 1990, which is incorporated by reference herein in its entirety;

(c) (dichloromethylene)-bis-phosphonic acid (clodronic acid) and the disodium salt (clodronate), which are described in Belgium Patent 672,205 (1966) and J. Org. Chem. 32: 4111 (1967), both of which are incorporated by reference herein in their entirety;

(d) [1-hydroxy-3-(1-pyrrolidinyl)-propylidene]-bis-phosphonate (EB-1053);

(e) (1-hydroxyethylidene)-bis-phosphonate (etidronate);

(f) [1-hydroxy-3-(methylpentylamino)propylidene]-bis-phosphonate (ibandronate), which is described in U.S. Pat. No. 4,927,814, issued May 22, 1990, which is incorporated by reference herein in its entirety;

(g) (6-amino-1-hydroxyhexylidene)-bis-phosphonate (neridronate);

(h) [3-(dimethylamino)-1-hydroxypropylidene]-bis-phosphonate (olpadronate);

(i) (3-amino-1-hydroxypropylidene)-bis-phosphonate (pamidronate);

(j) [2-(2-pyridinyl)ethylidene]-bis-phosphonate (piridronate), which is described in U.S. Pat. No. 4,761,406, which is incorporated by reference in its entirety;

(k) [1-hydroxy-2-(3-pyridinyl)-ethylidene]-bis-phosphonate (risedronate);

(l) {[(4-chlorophenyl)thio]methylene}-bis-phosphonate (tiludronate), which is described in U.S. Pat. No. 4,876,248, to Breliere et al., Oct. 24, 1989, which is incorporated by reference herein in its entirety;

(m) [1-hydroxy-2-(1H-imidazol-1-yl)ethylidene]-bis-phosphonate (zoledronate); and

(n) [1-hydroxy-2-imidazopyridin-(1,2-a)-3-ylethylidene]-bis-phosphonate (minodronate).

In one embodiment of the methods and compositions of the present invention, the bisphosphonate is selected from alendronate, clodronate, etidronate, ibandronate, incadronate, minodronate, neridronate, olpadronate, pamidronate, piridronate, risedronate, tiludronate, zoledronate, pharmaceutically acceptable salts thereof, and mixtures thereof. In a class of this embodiment, the bisphosphonate is selected from alendronate, risedronate, zoledronate, ibandronate, tiludronate, and clodronate. In a subclass of this class, the bisphosphonate is alendronate, pharmaceutically acceptable salts and hydrates thereof, and mixtures thereof. A particular pharmaceutically acceptable salt of alendronate is alendronate monosodium. Pharmaceutically acceptable hydrates of alendronate monosodium include the monohydrate and the trihydrate. A particular pharmaceutically acceptable salt of risedronate is risedronate monosodium. Pharmaceutically acceptable hydrates of risedronate monosodium include the hemi-pentahydrate.

As used throughout this specification and claims, the term “alendronic acid” includes the related bisphosphonic acid forms, pharmaceutically acceptable salt forms, and equilibrium mixtures of these. The term “alendronic acid” includes crystalline, hydrated crystalline, and amorphous forms of alendronic acid and pharmaceutically acceptable salts thereof. It specifically includes anhydrous alendronate monosodium, alendronate monosodium monohydrate, and alendronate monosodium trihydrate.

“Pharmaceutically acceptable salts and derivatives” of the bisphosphonates are also useful herein. Nonlimiting examples of salts include alkali metal, alkaline metal, ammonium, mono-(C₁-C₃₀)-alkyl-substituted ammonium, di-(C₁-C₃₀)-alkyl-substituted ammonium, tri-(C₁-C₃₀)-alkyl-substituted ammonium, and tetra-(C₁-C₃₀)-alkyl-substituted ammonium. In one embodiment the salts are those selected from sodium, potassium, calcium, magnesium, and ammonium salts. Nonlimiting examples of derivatives include those selected from esters and amides. Also encompassed within the scope of the present invention are the various hydrates and other solvates of the bisphosphonates, or a pharmaceutically acceptable salt thereof. For alendronate, these can include alendronate monosodium trihydrate, alendronate monosodium monohydrate, alendronate monosodium hemihydrate, anhydrous alendronate dipotassium, and alendronate dipotassium pentahydrate.

“Pharmaceutically acceptable” as used herein means that the salts and derivatives of the bisphosphonates that have the same general pharmacological properties as the free acid form from which they are derived and are acceptable from a toxicity viewpoint.

Methods of the Present Invention

The present invention comprises methods for treating or preventing a bone disorder in a mammal in need thereof comprising orally administering to said mammal a pharmaceutically effective amount of a liquid pharmaceutical composition of at least one, or a pharmaceutically acceptable salt thereof, pharmaceutically acceptable salt thereof, as a unit dosage according to a dosing schedule having a dosing interval selected from once-weekly dosing, twice-weekly dosing, biweekly dosing, twice-monthly dosing, and once-monthly dosing.

In one embodiment of the invention, the mammal is a human.

In a class of the invention the bone disorder is selected from selected from Paget's disease, osteoporosis, metastatic bone disease, hypercalcemia of malignancy, periprosthetic osteolysis, periodontal disease and arthritic conditions.

In a subclass of the invention the bone disorder is osteoporosis. In another subclass of the invention the bone disorder is Paget's disease. In another subclass of the invention the bone disorder is metatstatic bone disease. In another subclass of the invention the bone disorder is arthritic conditions. In an embodiment of that subclass, the bone disorder is osteoarthritis.

In another class of the invention the bisphosphonate is selected from selected from the group consisting of alendronate, clodronate, etidronate, ibandronate, incadronate, minodronate, neridronate, olpadronate, pamidronate, piridronate, risedronate, tiludronate, and zoledronate, or a pharmaceutically acceptable salts thereof, and mixtures thereof. In a subclass, the bisphosphonate is selected from alendronate, pharmaceutically acceptable salts thereof, and mixtures thereof. In a further subclass, the pharmaceutically acceptable salt selected from alendronate monosodium and hydrates thereof. In a further subclass, the hydrate of alendronate monosodium is the trihydrate or the monohydrate.

In an embodiment of the invention the liquid pharmaceutical composition is an aqueous solution. In a further embodiment, the unit dosage of alendronate ranges from about greater than 140 mg to about 1120 mg, on an alendronic acid active basis.

The present invention comprises methods for inhibiting abnormal bone remodeling in mammals with Paget's disease, osteoporosis, such as for example, osteoporosis in men, postmenopoausal women, and patients treated with glucocorticoid, metastatic bone disease, hypercalcemia of malignancy, periprosthetic osteolysis, periodontal disease and arthritic conditions. The present invention also comprises treating abnormal bone remodeling in mammals with Paget's disease, metastatic bone disease, hypercalcemia of malignancy, periprosthetic osteolysis, periodontal disease and arthritic conditions. The present invention also comprises methods for preventing abnormal bone remodeling in mammals with Paget's disease, osteoporosis, such as for example, osteoporosis in men, postmenopoausal women, and patients treated with glucocorticoid, metastatic bone disease, hypercalcemia of malignancy, periprosthetic osteolysis, periodontal disease and arthritic conditions. In one embodiment of the invention, the mammal is a human.

The present invention also comprises methods for inhibiting abnormal bone resorption in mammals with Paget's disease, osteoporosis such as for example, osteoporosis in men, postmenopoausal women, and patients treated with glucocorticoid, metastatic bone disease, hypercalcemia of malignancy, periprosthetic osteolysis, periodontal disease and arthritic conditions. The present invention also comprises treating abnormal bone resorption in mammals with Paget's disease, osteoporosis such as for example, osteoporosis in men, postmenopoausal women, and patients treated with glucocorticoid, metastatic bone disease, hypercalcemia of malignancy, periprosthetic osteolysis, periodontal disease, and arthritic conditions. The present invention also comprises methods for preventing abnormal bone resorption in mammals with Paget's disease, osteoporosis, such as for example, osteoporosis in men, postmenopoausal women, and patients treated with glucocorticoid, metastatic bone disease, hypercalcemia of malignancy, periprosthetic osteolysis, periodontal disease and arthritic conditions. In one embodiment of the mammal is a human.

The present invention comprises a continuous dosing schedule whereby a unit dosage of a high dose buffered liquid bisphosphonate formulation is regularly administered according to a dosing interval selected from the group consisting of once-weekly dosing, twice-weekly dosing, biweekly dosing, twice-monthly dosing and once-monthly dosing.

By once-weekly dosing it is meant that a unit dosage of a high dose buffered liquid bisphosphonate formulation is administered once a week, i.e. one time during a seven day period, such as for example, on the same day of each week. In the once-weekly dosing regimen, the unit dosage is generally administered about every seven days. A nonlimiting example of a once-weekly dosing regimen would entail the administration of a unit dosage of a buffered liquid bisphosphonate formulation every Sunday. In one embodiment, the unit dosage is not administered on consecutive days, but the once-weekly dosing regimen can include a dosing regimen in which unit dosages are administered on two consecutive days falling within two different weekly periods.

By twice-weekly dosing it is meant that a unit dosage of a buffered liquid bisphosphonate is administered twice a week, i.e. two times during a seven day period, such as for example, on the same two days of each weekly period. In the twice-weekly dosing regimen, each unit dosage is generally administered about every three to four days. A nonlimiting example of a twice-weekly dosing regimen would entail the administration of a unit dosage of a buffered liquid bisphosphonate formulation every Sunday and Wednesday. In one embodiment, the unit dosages are not administered on the same or consecutive days, but the twice-weekly dosing regimen can include a dosing regimen in which unit dosages are administered on two consecutive days within a weekly period or different weekly periods.

By biweekly dosing it is meant that a unit dosage of a buffered liquid bisphosphonate formulation is administered once during a two week period, i.e. one time during a fourteen day period, such as for example, on the same day during each two week period. In the twice-weekly dosing regimen, each unit dosage is generally administered about every fourteen days. A nonlimiting example of a biweekly dosing regimen would entail the administration of a unit dosage of a buffered liquid bisphosphonate formulation every other Sunday. In one embodiment, the unit dosage is not administered on consecutive days, but the biweekly dosing regimen can include a dosing regimen in which the unit dosage is administered on two consecutive days within two different biweekly periods.

By twice-monthly dosing it is meant that a unit dosage of a buffered liquid bisphosphonate formulation is administered two times, during a monthly calendar period. With the twice-monthly regimen, the doses are given, for example, on the same two dates of each month. In the twice-monthly dosing regimen, each unit dosage is generally administered about every fourteen to sixteen days. A nonlimiting example of a twice-monthly dosing regimen would entail dosing on or about the first of the month and on or about the fifteenth, i.e., the midway point, of the month. In one embodiment, the unit dosages are not administered on the same or consecutive days but the twice-monthly dosing regimen can include a dosing regimen in which the unit dosages are administered on two consecutive days within a monthly period, or different monthly periods. The twice-monthly regimen is defined herein as being distinct from, and not encompassing, the biweekly dosing regimen because the two regimens have a different periodicity and result in the administration of different numbers of dosages over long periods of time. For example, over a one year period, a total of about twenty-four dosages would be administered according to the twice-monthly regimen (because there are twelve calendar months in a year) whereas a total of about twenty-six dosages would be administered according to the biweekly dosing regimen (because there are about fifty-two weeks in a year).

By once-monthly dosing it is meant that a unit dosage of a buffered liquid bisphosphonate formulation is administered once a month, i.e. one time during a 28, 30, or 31 day period, such as for example, on the same date of each month. A nonlimiting example of a once-monthly dosing regimen would entail the administration of a unit dosage of a buffered liquid bisphosphonate formulation on the first day of every month. In one embodiment, the unit dosage is not administered on consecutive days, but the once-monthly dosing regimen can include a dosing regimen in which unit dosages are administered on two consecutive days falling within two different months, e.g. January 31^{th} and February 1^{st}. Therefore, over a one year period, a total of about twelve dosages would be administered according to the once-monthly regimen (because there are twelve calendar months in a year). In the monthly dosing regimen, each unit dosage can typically be administered about every twenty-eight to thirty-five days.

The methods and compositions of the present invention are useful for inhibiting abnormal bone remodeling and for treating and preventing abnormal bone remodeling and other bone pathologies associated with Paget's disease, osteoporosis such as for example, osteoporosis in men, postmenopoausal women, and patients treated with glucocorticoid, metastatic bone disease, hypercalcemia of malignancy, periprosthetic osteolysis, and arthritic conditions. Such conditions can include, as in Paget's disease, excessive bone resorption and bone formation leading to increased bone remodeling, bone hypertrophy, and abnormal bone structure. Clinical consequences include bone pain, bone enlargement, bone deformity, and increased blood flow; secondary complications include osteoarthritic bone pain due to deformity of bones adjacent to joints, entrapment of nerves, especially if the spine or the base of the skull is affected.

Because of the mixed nomenclature currently in use by those of ordinary skill in the art, reference to a specific weight or percentage of alendronate in the present invention is on an acid active weight basis, unless indicated otherwise herein. For example, the phrase “about 280 mg of alendronate, or pharmaceutically acceptable salt thereof, on an alendronic acid active weight basis” means that the amount of alendronate is calculated based on 280 mg of alendronic acid. Even though it is conventional to dose and calculate the dosages of bisphosphonates on the basis of bisphosphonic acid active weight basis, bisphosphonate dosages can be calculated and administered based on other salt or hydrate forms. For example, risedronate dosages are calculated based on the weight of the anhydrous risedronate sodium salt. Bisphosphonate doses calculated on the basis of their salt, derivative or hydrate forms are included within the dosage ranges of the present invention on the basis of their bisphosphonic acid active weights. Additionally, the doses of all hydrate forms of alendronate are calculated on the basis of the alendronic acid active weight. For instance, the doses of the monohydrate, trihydrate, hemihydrate and all other hydrate forms of alendronate and its salts, are calculated on the basis of their alendronic acid active weights. Reference to a specific weight or percentage of risedronate is on an anhydrous risedronate monosodium active weight basis, unless otherwise indicated. For example, the phrase “about 210 mg of risedronate on an anhydrous risedronate monosodium active weight basis” means that the amount of risedronate is calculated based on 210 mg of anhydrous risedronate monosodium. In one embodiment of the invention, the form of risedronate is risedronate monosodium hemi-pentahydrate with small amounts of monohydrate.

Pharmaceutical Compositions

Another aspect of the present invention is concerned with oral liquid pharmaceutical compositions of bisphosphonates as unit dosages for administration according to a continuous schedule having a dosing interval chosen from once-weekly, twice-weekly, biweekly, twice-monthly, and once-monthly.

The oral liquid pharmaceutical composition in accordance with this invention comprises: a) a therapeutically effective amount of at least one or a pharmaceutically acceptable salt thereof, b) a pharmaceutically acceptable carrier, and c) a pharmaceutically acceptable buffer. A dose of the oral liquid pharmaceutical composition has a buffering capacity sufficient to buffer 50 mL of 0.1 N HCl to a pH of at least 3.5.

In one embodiment of the invention, the at least one bisphosphonate is alendronate or a pharmaceutically acceptable salt thereof.

A dose volume of the composition can range from about 30 mL to about 150 mL, such as for example, from about 75 to about 100 mL. In one embodiment of the invention, the oral liquid composition is capable of raising the pH of about 100 mL of simulated stomach acid (0.1 N HCl) to a pH of about 3.5 or greater.

The oral liquid pharmaceutical composition in accordance with the present invention has a pH that falls within a range physiologically suitable for oral administration. In one embodiment of the invention, the oral liquid pharmaceutical composition has a pH ranging from about 5 to about 10, such as for example, from about 6.4 to about 7.2, further still from about 6.6 to about 7.0.

It has been found that administration of at least one via a buffered formulation having a specific buffering capacity minimizes the occurrence of or potential for adverse gastrointestinal effects. The use of a buffering agent reduces the requirement that the patient also take various additional antacids to combat acid reflux, heartburn, or other gastric problems.

The type of pharmaceutically acceptable buffer, which can be used in the oral liquid pharmaceutical composition of the present invention, has a buffering capacity such that a relatively small volume can raise the pH of stomach acid sufficiently. Non-limiting examples of buffering systems that can be employed include citrate, tartrate, fumarate, phosphate, succinate, acetate, and mixtures thereof.

In one embodiment of the invention, the pharmaceutically acceptable buffer is selected from citrate and tartrate. In yet another embodiment, the buffer is citrate. In one variant, a 75 mL dose of the formulation of this invention will contain a sufficient amount of one or more of the following compounds chosen from disodium hydrogenphosphate, potassium dihydrogenphosphate, sodium hydrogenphthalate, trisodium citrate dihydrate, disodium hydrogen tartrate dihydrate, and disodium formate, such that the final formulation will be capable of buffering about 100 mL of 0.1 N HCl to a pH of about 3.5 or greater. For example, a useful buffering system is a citrate buffer comprising trisodium citrate dihydrate, present at about 72 mM.

In addition to the foregoing buffers, various bases, such as sodium hydroxide, potassium hydroxide, ammonium hydroxide, and the like, can be used to further adjust the pH of the buffer system in an upward direction. Alternatively, an aqueous acid, such as aqueous hydrochloric acid, can be used to further adjust the pH of the buffer system in a downward direction.

The ability of the formulation to buffer about 50 mL to about 100 mL of stomach acid is important to minimize the negative effects of reflux of gastric contents into the esophagus. Reflux of gastric contents can cause irritation, and even erosion of the esophagus. Typically, a person has about 50 mL of gastric juice in the stomach having a pH of about 1. Upon taking the alendronic acid formulation of the present invention, the stomach acid is buffered to at least 3.5, such as for example, to about 4. It has been found, in animal models in accordance with this invention, that an alendronate sodium and gastric acid mixture at a pH of at least 3.5 is not irritating to esophageal tissues. Thus, if the patient experiences reflux, the chance of esophageal irritation will be minimal.

The oral liquid formulations optionally contain at least one additional agent. Non-limiting examples of the at least one additional agent are chosen from stabilizers, flavoring agents, colorants, and sweeteners. The inclusion of a flavoring agent and/or sweetener in the liquid dosage formulation provides for improved patient compliance.

In one embodiment of the invention, the formulation further comprises at least one form of vitamin D. The term “vitamin D,” as used herein, means non-activated forms of both vitamin D₂ (ergocalciferol) and vitamin D₃ (cholecalciferol). The phrase “a non-activated metabolite of vitamin D₂ and/or D₃,” as used herein, means hydroxylated forms of vitamin D₂ (ergocalciferol) and vitamin D₃ (cholecalciferol), e.g. 25-hydroxy-cholecalciferol, and 24,25-dihydroxy-cholecalciferol. These non-activated metabolites are the primary storage form of vitamin D₃ in the human body. 25-Hydroxy-cholecaliferol may be further hydroxylated in the body to form 1,25-dihydroxy-cholecalciferol (an active precursor of vitamin D₃).

The term “IU,” as used herein, means International Units. One microgram of vitamin D is approximately 40 International Units. For instance, 1 IU (or USP) of vitamin D₃ is defined as the activity of 0.025 micrograms of vitamin D₃. For example, 2800 IU of vitamin D₃ is equivalent to about 70 micrograms of vitamin D₃. Since 25-hydroxyvitamin D₃ is about 1.4 times (1.4×) more potent than vitamin D₃, 50 micrograms of 25-hydroxyvitamin D₃ would be equivalent to about 70 micrograms (2800 IU) of vitamin D₃.

The term “supplementary effective amount,” as used herein, means an exogenous amount of vitamin D₂ and/or D₃ metabolites, wherein the amount is sufficient for reducing vitamin D deficiency in mammals, and particularly mammals suffering from abnormal bone resorption. Generally, an appropriate supplementary amount of a non-activated metabolite of vitamin D₂ and/or D₃ may be chosen to supplement vitamin D nutrition during the dosing interval. For humans, the supplementary amount of non-activated metabolite of vitamin D₂ and/or D₃ may range from about 100 to about 60,000 IU/day; typically, the supplementary amount of a non-activated metabolite of vitamin D₂ and/or D₃ may range from about 200 to about 40,000 IU/day; such as for example from about 400 to about 1,200 IU/day. However, the maximum amount of vitamin D generally should not exceed about 100,000 IU/administration. As high as 4,000 IU/day, as well as, doses higher than 4,000 IU may be given at less frequent intervals due to the long body half-life of vitamin D (See Vieth R. et al., Efficacy and safety of vitamin D₃ intake exceeding the lowest observed adverse effect level, Am J Clin Nutr (2001); 73; 288-94; and Vieth R. Vitamin D Supplementation, 25-hydroxy vitamin D concentration, and safety, Am J Clin Nutr 1999; 69: 842-856). Unless otherwise noted, all vitamin D dosages are based on the interval, i.e. days, between dosing. For example, if 200 IU/day of vitamin D is desired and the dosing period is weekly (based on 7 days/week), the weekly dosing would be 200 IU/day ×7 days/week or 1,400 IU/week of vitamin D.

For humans, compositions characterized as containing alendronate, pharmaceutically acceptable salts, derivatives and hydrates thereof, and a non-activated metabolite of vitamin D₂ and/or D₃, a general oral supplemental amount of a non-activated metabolite of vitamin D₂ and/or D₃ containing from about 100 IU to about 60,000 IU are contemplated. Non-limiting examples of an oral supplementary amount of a non-activated metabolite of vitamin D₂ and/or D₃ include, but are not limited to, dosages of 1,400 IU, 2,800 IU, 4,200 IU, 5600 IU, 7,000 IU, 8,400 IU, 14,000 IU, and 28,000 IU in combination with varying amounts of at least one bisphosphonate.

In one embodiment of the invention, for humans, the oral pharmaceutical composition includes a non-activated metabolite of vitamin D₂ and/or D₃ and a pharmaceutically effective amount of alendronate, pharmaceutically acceptable salts, derivatives and hydrates thereof, typically containing from greater than about 140 mg to about 1120 mg, on an alendronic acid active weight basis. Non-limiting examples of a pharmaceutically effective amount of alendronate include, but are not limited to, dosages of about 160 mg, 280 mg, 375 mg, 560 mg, 800 mg, 1000 mg, and 1120 mg of alendronate, on an alendronic acid active weight basis, pharmaceutically acceptable salts, derivatives, hydrates, and mixtures thereof, in combination with varying amounts of a non-activated metabolite of vitamin D₂ and/or D₃.

The formulation can optionally contain a preservative system comprising at least one preservative of adequate type and concentration to control potential microbial contamination. Preservatives that can be used to provide multiple dosing can be any of the known pharmaceutically acceptable preservatives which are active in a liquid having a pH of ranging from about 3.5 to about 10.0, such as for example, ranging from about 4 to about 8, and which do not adversely interact with the active ingredient, at least one, such as alendronate. Such preservatives should be used at their usual concentrations. Non-limiting representatives of preservatives include: benzoic acid and its pharmaceutically acceptable salts, for example, sodium benzoate, and potassium benzoate; sorbic acid and its pharmaceutically acceptable salts, for example, potassium sorbate, parabens and their pharmaceutically acceptable salts, and alcohols such as ethanol and benzyl alcohol.

Non-limiting examples of parabens and pharmaceutically acceptable salts of parabens include sodium propylparaben, sodium butyl paraben, methylparaben, sodium methylparaben, ethylparaben, propylparaben, and butylparaben. In one embodiment of the invention, the preservative system comprises at least one compound chosen from sodium propylparaben and sodium butylparaben. The concentration of sodium propylparaben and sodium butylparaben in the formulation can range from 0.12-0.5 mg/mL mg/mL and 0.04-0.2 mg/mL, respectively.

In one embodiment of the invention, the oral liquid pharmaceutical composition of alendronate on an alendronic acid active weight basis comprises the following formula:

| Ingredient | Concentration |
|---|---|
| Alendronate monosodium | 1.86-17.8 mg/mL |
| trihydrate* | |
| Citrate | 72 mM |
| Sodium propylparaben | 0.12-0.5 mg/mL |
| Sodium butylparaben | 0.04-0.2 mg/mL |
| Sodium saccharin | 0-5 mg/mL |
| NaOH | pH adjustment (pH 3-10) |
| HCl | pH adjustment (pH 3-10) |
| Water | Qs 1 mL |

*Corresponds to 1.86-17.8 mg/mL free alendronic acid equivalents/mL
The total dose volume of the above formulation is large enough to ensure that the total amount of alendronate present in the dose ranges from greater than about 140 mg to about 1335 mg on an alendronic acid active weight basis.

It can also be desired to include at least one taste enhancer, such as sweeteners although their inclusion in the formulation is strictly optional. Examples of suitable pharmaceutically acceptable sweeteners include aspartame, acesulfame-K, saccharin and its pharmaceutically acceptable salts, dextrose, glycerin, maltose, galactose, dextrose, sorbitol, xylitol, fructose, lactose, and sucrose. A useful concentration of sorbitol or xylitol is between about 20 and 140 mg/mL, such as for example, from about 40 to about 100 mg/mL. Sodium saccharin is another useful sweetener and can be used at a concentration ranging of about 0.02 to about 5 mg/mL, such as for example, concentrations up to about 0.1 mg/mL. Combinations of one or more sweeteners can also be used.

Additional optional ingredients include at least one agent chosen from pharmaceutically acceptable flavoring agents and pharmaceutically acceptable colorants. Flavoring agents which have been found to be suitable are, but are not limited to, peach, berry, strawberry, all citrus flavors, tomato, apple, tutti-frutti and mixtures thereof. Examples of typical concentrations are 2-10 mg/mL, suitable to provide acceptable flavor. Non-limiting examples of pharmaceutically acceptable colorants include FD&C Blue 2, FD&C Red 33. Such coloring agents and flavoring agents can be employed in their usual concentrations.

A further additional optional ingredient is a stabilizer, such as EDTA (ethylenediaminetetraacetic acid).

The pharmaceutically acceptable carrier used in accordance with this invention can be any usual and known pharmaceutical media can be employed, such as for example, water or glycols. The oral liquid pharmaceutical composition of the present invention can be a syrup or an elixir formulated with sweetening agents, for example, glycerol, sorbitol, or sucrose.

In one embodiment, the oral liquid pharmaceutical composition is an effervescent composition.

In one embodiment of the invention when the at least one bisphosphonate is alendronate, the concentration of alendronate or a pharmaceutically acceptable salt thereof ranges from about 1.86 mg/mL to about 17.8 mg/mL, on an alendronic acid active weight basis.

The precise dosage of the at least one bisphosphonate will vary with the dosing schedule, the oral potency of the particular bisphosphonate chosen, the age, size, sex, and condition of the mammal or human, the nature and severity of the disorder to be treated, and other relevant medical and physical factors. Thus, the precise pharmaceutically effective amount can readily be determined by the caregiver or clinician. Routine experimentation from animal models and human clinical studies can determine appropriate amounts. Generally, an appropriate amount of bisphosphonate is chosen to obtain an abnormal bone remodeling inhibiting effect, i.e., an abnormal bone remodeling inhibiting amount is administered. For humans, an effective oral liquid dose of bisphosphonate is typically from about 0.01 to about 100 mg/kg body weight and, such as for example, about 0.1 to about 20 mg/kg of body weight.

Examples of a twice-weekly dosing include oral unit dosages which are useful for treatment of a condition chosen from osteoporosis, and osteoarthritis, comprising greater than about 140 mg of the alendronate compound, on an alendronic acid active weight basis, in an aqueous solution, and in one embodiment, in about 75 mL of a buffered aqueous solution.

Another example of a twice-weekly dosing includes an oral unit dosage which is useful for treatment or prevention of metastatic bone disease comprising greater than about 140 mg of the alendronate compound, on an alendronic acid active weight basis, in an aqueous solution, and in one embodiment, in about 75 mL of a buffered aqueous solution.

Yet another example of a twice-weekly dosing includes an oral unit dosage which is useful for treatment of Paget's Disease comprising about 280 mg of the alendronate compound, on an alendronic acid active weight basis, in an aqueous solution, and in one embodiment, in about 75 mL of a buffered aqueous solution.

Examples of a once-weekly dosing include oral unit dosages which are useful for treatment of a condition chosen from osteoporosis, and osteoarthritis, comprising from about 280 mg of the alendronate compound, on an alendronic acid active weight basis, in an aqueous solution, and in one embodiment, in about 75 mL of a buffered aqueous solution.

Another example of a once-weekly dosing includes an oral unit dosage which is useful for treatment or prevention of metastatic bone disease comprising from about 280 mg of the alendronate compound, on an alendronic acid active weight basis, in an aqueous solution, and in one embodiment, in about 75 mL of a buffered aqueous solution.

Yet another example of a once-weekly dosing includes an oral unit dosage which is useful for the treatment of Paget's Disease ranging from about 280 mg to about 560 mg of the alendronate compound, on an alendronic acid active weight basis, in an aqueous solution, and in one embodiment, in about 75 mL of a buffered aqueous solution.

An example of biweekly dosing or twice-monthly dosing includes oral unit dosages useful for treating osteoporosis comprising about 280 mg of the alendronate compound, on an alendronic acid active weight basis, in an aqueous solution, and in one embodiment, in about 75 mL of a buffered aqueous solution.

Other example of biweekly dosing or twice-monthly dosing includes an oral unit dosage useful for treating or preventing metastatic bone disease comprising about 560 mg of the alendronate compound, on an alendronic acid active weight basis, in an aqueous solution, and in one embodiment, in about 75 mL of a buffered aqueous solution.

Other example of biweekly dosing or twice-monthly dosing includes an oral unit dosage useful for treating osteoarthritis comprising about 560 mg of the alendronate compound, on an alendronic acid active weight basis, in an aqueous solution, and in one embodiment, in about 75 mL of a buffered aqueous solution.

Yet another example of biweekly dosing or twice-monthly dosing includes oral unit dosages useful for treating Paget's Disease comprising from about 280 to about 1120 mg, such as for example, 560 mg, of the alendronate compound, on an alendronic acid active weight basis, in an aqueous solution, and in one embodiment, in about 75 mL of a buffered aqueous solution.

An example of once-monthly dosing includes oral unit dosages useful for treating Paget's Disease comprising from about 280 mg to about 1120 mg, such as for example, 375 mg or 560 mg, of the alendronate compound, on an alendronic acid active weight basis, in an aqueous solution, and in one embodiment, in about 75 mL of a buffered aqueous solution.

Other examples of once-monthly dosing include oral unit dosages useful for treating osteoarthritis comprising from about 560 mg to about 1120 mg of the alendronate compound, on an alendronic acid active weight basis, in an aqueous solution, and in one embodiment, in about 75 mL of a buffered aqueous solution.

Additional examples of once-monthly dosing include oral unit dosages useful for treating or preventing metastatic bone disease comprising from about 560 mg to about 1120 mg of the alendronate compound, on an alendronic acid active weight basis, in an aqueous solution, and in one embodiment, in about 75 mL of a buffered aqueous solution.

Further examples of once-monthly dosing include oral unit dosages useful for treating osteoporosis comprising from about 280 mg to about 560 mg of the alendronate compound, on an alendronic acid active weight basis, in an aqueous solution, and in one embodiment, in about 75 mL of a buffered aqueous solution.

At dose concentrations of 140 mg and higher, the formulations of the oral buffered solution exhibit nonlinear dose proportionality. Suprisingly, the bioavailability of these high dose formulations is greater than expected. The absolute oral bioavailability of alendronate is measured by comparing the 36-hour urinary excretion of alendronate after a single oral dose to that after a reference dose of intravenous alendronate dose. The relative oral bioavailability of two different oral doses of alendronate is measured by comparing the 36-hour urinary excretion after these oral doses.

Relative to an intravenous (IV) reference dose, the mean oral bioavailability of alendronate in women was 0.64% for doses ranging from 5- to 70-mg when administered after an overnight fast and two hours before a standardized breakfast. Oral bioavailability of the 10-mg tablet in men (0.59%) was similar to that in women when administered after an overnight fast and 2 hours before breakfast. Bioavailability was decreased (by approximately 40%) when 10-mg alendronate was administered either 0.5 or 1 hour before a standardized breakfast, when compared to dosing 2 hours before eating. See Physician's Desk Reference, (55^{th} Edition, page 1930, (2001).

The oral bioavailability of oral alendronate tablets, in doses ranging from 5- to 80-mg, in comparison to an intravenous dose of 0.125- or 0.25-mg, was similar throughout the dose range. The bioavailability of alendronate in postmenopausal women averaged about 0.76% of the oral dose. The bioavailability in men averaged about 0.6%. Results of this study are reported in the following publication, incorporated by reference herein, A. G. Porras, et al., “Pharmacokinetics of Alendronate,” Clin Pharmacokinet, 36 (5): 315-328.

A study was conducted to compare the urinary excretion of alendronate following a 70-mg alendronate oral buffered solution to that observed following an alendronate 70-mg tablet. The relative bioavailability of the 70-mg oral buffer solution compared to a 70-mg alendronate tablet was determined to be 1.01. Thus indicating the bioequivalence of the 70-mg oral buffer solution to the 70-mg tablet.

Surprisingly, however, when alendronate was administered as an oral buffered solution, nonlinear dose proportionality was observed at doses 140-mg and higher. Relative to the 70-mg dose of the oral buffered solution, the relative urinary excretions were increased 1.65, 1.97, and 1.68 fold, for the 140-, 280- and 375-mg doses of the oral buffered solution, respectively, consistent with a higher bioavailability at a dose of 140-mg or higher. These results are confirmed by an absolute oral bioavailability study. In this study, the oral bioavailability of a 280-mg alendronate oral buffered solution was compared to the bioavailability of a reference 1-mg intravenous dose of alendronate. The absolute oral bioavailability of the 280-mg oral buffered solution was found to be 0.86%. This is approximately 34% higher than the 0.64% observed with the alendronate tablet formulation.

This increased oral bioavailability is of particular clinical importance. The toxicity associated with oral bisphosphonates is primarily due to its local effect on the upper gastrointestinal mucosa. The local toxicity could be reduced, while maintaining efficacy, by administering a higher dose of the bisphosphonate at a less frequent dosing interval. For example, instead of administering a bisphosphonate daily, seven times the daily dose could be administered once weekly. However, as with any medication, toxicity increases with increasing dose and there is an upper limit as to the dose that can be administered, even with a less frequent dosing regimen. The present finding of a higher bioavailability of alendronate at doses 140-mg and above, administered in an oral buffered formulation, facilitates the treatment of conditions that require higher doses of alendronate to achieve optimal efficacy, such as, Paget's disease, metastatic bone disease, hypercalcemia of malignancy, periprosthetic osteolysis, periodontal disease and arthritic conditions. The formulation of present invention allows the administration of higher systemic (bioavailable) doses with a disproportionally lower dose. This will result in an improved efficacy/tolerability ratio.

Other non-limiting examples of oral compositions in buffered aqueous solutions comprising alendronate, as well as other bisphosphonates, are illustrated in the Examples below.

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

### Example 1

A liquid formulation containing about 141 mg of alendronate monosodium trihydrate, on an alendronic acid active basis, in about 75 mL of liquid was prepared using the following relative weights of ingredients:

| Ingredient | Concentration |
|---|---|
| Alendronate monosodium trihydrate* | 2.454 mg/mL |
| Sodium Citrate dihydrate | 21.18 mg/mL |
| NaOH | qs pH 6.8 |
| HCl | qs pH 6.8 |
| Water | qs 1.00 mL |

*Corresponds to 1.88 mg free alendronic acid equivalents or 141 mg/75 mL

Additional agents such as cosolvents, flavoring agents, coloring agents, preservatives, and stabilizers can also be specifically incorporated in the formulation as follows:

| | |
|---|---|
| Alendronate monosodium trihydrate* | 2.454 mg/mL |
| Sodium Citrate | 21.18 mg/mL |
| Sodium propylparaben | 0.23 mg/mL |
| Sodium butyl hydroxybenzoate | 0.07500 mg/mL |
| Sodium saccharin | 0.1000 mg/mL |
| Raspberry flavor | qs for taste |
| Sodium hydroxide | qs pH 6.8 |
| Hydrochloric acid | qs pH 6.8 |
| Purified Water | qs 1.00 mL |

*Corresponds to 1.88 mg free alendronic acid equivalents or 140 mg/75 mL

Method of Manufacture:

The specified amounts of the above ingredients were added to purified water and mixed sequentially until dissolved: sodium propylparaben, sodium butylparaben, sodium citrate dihydrate, sodium saccharin, raspberry flavor, and alendronate monosodium trihydrate. The pH was checked to target about pH 6.8 (range: about 6.4 to about 7.2). If required, the pH was adjusted to 6.8 with aqueous sodium hydroxide or aqueous hydrochloric acid. The batch was then adjusted to final weight with purified water and filtered through a suitable filter (<50 μm). The solution was then dispensed into suitable containers and capped. The aqueous solution can be used as is directly from the bottle or poured into a suitable container for dosing.

### Example 2

Twice-Weekly Dosing Regimen for Treatment of Paget's Disease of Bone

The alendronate formulation from Example 1 containing about 141 mg of alendronate in about 75 mL of liquid, on an alendronic acid active basis, is orally administered to a human patient twice-weekly, such as , once every three or four days (for example every Sunday and Wednesday), for a period of at least one to six months. This method of administration is useful and convenient for treating Paget's disease of bone, particularly in humans with difficulty in swallowing tablets, while minimizing adverse upper gastrointestinal effects, particularly esophageal irritation. This method is also useful for improving patient acceptance and compliance.

### Example 3

Twice-Weekly Dosing Regimen for Treatment of Osteoporosis

The alendronate formulation from Example 1 containing about 141 mg of alendronate in about 75 mL of liquid, on an alendronic acid active basis, is orally administered to a human patient twice-weekly, such as, once every three or four days (for example, every Sunday and Wednesday), for a period of at least one to six months. This method of administration is useful and convenient for treating Osteoporosis, particularly in humans with difficulty in swallowing tablets, while minimizing adverse upper gastrointestinal effects, particularly esophageal irritation. This method is also useful for improving patient acceptance and compliance.

### Example 4

Twice-Weekly Dosing Regimen for Treatment of Osteoarthritis

The alendronate formulation from Example 1 containing about 141 mg of alendronate in about 75 mL of liquid, on an alendronic acid active basis, is orally administered to a human patient twice-weekly, such as once every three or four days (for example, every Sunday and Wednesday), for a period of at least one to six months. This method of administration is useful and convenient for treating osteoarthritis, particularly in humans with difficulty in swallowing tablets, while minimizing adverse upper gastrointestinal effects, particularly esophageal irritation. This method is also useful for improving patient acceptance and compliance.

### Example 5

Twice-Weekly Dosing Regimen for Treatment or Prevention of Metastatic Bone Disease

The alendronate formulation from Example 1 containing about 141 mg of alendronate in about 75 mL of liquid, on an alendronic acid active basis, is orally administered to a human patient twice-weekly, such as, once every three or four days (for example, every Sunday and Wednesday), for a period of at least one to six months. This method of administration is useful and convenient for treating or preventing metastatic bone disease in humans with lung, breast, and prostate cancer. The formulation is particularly beneficial in lung, breast, or prostate cancer patients who experience difficulty in swallowing tablets, while minimizing adverse upper gastrointestinal effects, particularly esophageal irritation.

### Example 6

Once-Weekly Dosing Regimen for Prevention of Osteoporosis

The alendronate formulation from Example 1 containing about 141 mg of alendronate in about 75 mL of liquid, on an alendronic acid active basis, is orally administered to a human patient once-weekly, such as, about once every seven days (for example, every Sunday), for a period of at least one to six months. This method of administration is useful and convenient for treating/preventing osteoporosis, particularly in humans with difficulty in swallowing tablets, while minimizing adverse upper gastrointestinal effects, particularly esophageal irritation. This method is also useful for improving patient acceptance and compliance.

### Example 7

A liquid formulation containing about 280 mg of alendronate monosodium trihydrate, on an alendronic acid active basis, in about 75 mL of liquid was prepared using the following relative weights of ingredients:

| Ingredient | Concentration |
|---|---|
| Alendronate monosodium trihydrate* | 4.873 mg/mL |
| Sodium Citrate dihydrate | 21.18 mg/mL |
| NaOH | qs pH 6.8 |
| HCl | qs pH 6.8 |
| Water | qs 1.00 mL |

*Corresponds to 3.733 mg free alendronic acid equivalents or 280 mg/75 mL

Additional agents such as cosolvents, flavoring agents, coloring agents, preservatives, and stabilizers can also be specifically incorporated in the formulation as follows:

| | |
|---|---|
| Alendronate monosodium trihydrate* | 4.873 mg/mL |
| Sodium Citrate | 21.18 mg/mL |
| Sodium propylparaben | 0.2250 mg/mL |
| Sodium butylparaben | 0.07500 mg/mL |
| Sodium saccharin | 0.1000 mg/mL |
| Raspberry flavor | qs for taste |
| Sodium hydroxide | qs pH 6.8 |
| Hydrochloric acid | qs pH 6.8 |
| Purified Water | qs 1.00 mL |

*Corresponds to 3.733 mg free alendronic acid equivalents or 280 mg/75 mL

The method of manufacture was the same as that for Example 1.

### Example 8

Twice-Weekly Dosing Regimen for Treatment of Paget's Disease of Bone

The alendronate formulation from Example 7 containing about 280 mg of alendronate in about 75 mL of liquid, on an alendronic acid active basis, is orally administered to a human patient twice-weekly, such as, once every three or four days (for example, every Sunday and Wednesday), for a period of at least one to six months. This method of administration is useful and convenient for treating Paget's disease of bone, particularly in humans with difficulty in swallowing tablets, while minimizing adverse upper gastrointestinal effects, particularly esophageal irritation. This method is also useful for improving patient acceptance and compliance.

### Example 9

Once-Weekly Dosing Regimen for Treatment of Paget's Disease of Bone

The alendronate formulation from Example 7 containing about 280 mg of alendronate in about 75 mL of liquid, on an alendronic acid active basis, is orally administered to a human patient once-weekly, such as, about once every seven days (for example, every Sunday), for a period of at least one to six months. This method of administration is useful and convenient for treating Paget's disease of bone, particularly in humans with difficulty in swallowing tablets, while minimizing adverse upper gastrointestinal effects, particularly esophageal irritation. This method is also useful for improving patient acceptance and compliance.

### Example 10

Biweekly Dosing Regimen for Treatment of Paget's Disease of Bone

The alendronate formulation from Example 7 containing about 280 mg of alendronate in about 75 mL of liquid, on an alendronic acid active basis, is orally administered to a human patient biweekly, such as, about once every fourteen days (for example, on alternate Sundays), for a period of at least one to six months. This method of administration is useful and convenient for treating Paget's disease of bone, particularly in humans with difficulty in swallowing tablets, while minimizing adverse upper gastrointestinal effects, particularly esophageal irritation. This method is also useful for improving patient acceptance and compliance.

### Example 11

Once-Monthly Dosing Regimen for Treatment of Paget's Disease with 280 mg Composition

The alendronate formulation from Example 7 containing about 280 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating Paget's disease, particularly in humans with difficulty in swallowing tablets, while minimizing adverse upper gastrointestinal effects, particularly esophageal irritation. This method is also useful for improving patient acceptance and compliance.

### Example 12

Once-Weekly Dosing Regimen for Treatment of Osteoporosis

The alendronate formulation from Example 7 containing about 280 mg of alendronate in about 75 mL of liquid, on an alendronic acid active basis, is orally administered to a human patient once-weekly, such as, about once every seven days (for example, every Sunday), for a period of at least one to six months. This method of administration is useful and convenient for treating osteoporosis, particularly in humans with difficulty in swallowing tablets, while minimizing adverse upper gastrointestinal effects, particularly esophageal irritation. This method is also useful for improving patient acceptance and compliance.

### Example 13

Biweekly Dosing Regimen for Treatment of Osteoporosis

The alendronate formulation from Example 7 containing about 280 mg of alendronate in about 75 mL of liquid, on an alendronic acid active basis, is orally administered to a human patient biweekly, such as, about once every fourteen days (for example, on alternate Sundays), for a period of at least one to six months. This method of administration is useful and convenient for treating osteoporosis, particularly in humans with difficulty in swallowing tablets, while minimizing adverse upper gastrointestinal effects, particularly esophageal irritation. This method is also useful for improving patient acceptance and compliance.

### Example 14

Once-Monthly Dosing Regimen for Treatment of Osteoporosis

The alendronate formulation from Example 7 containing about 280 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating osteoporosis, particularly in humans with difficulty in swallowing tablets, while minimizing adverse upper gastrointestinal effects, particularly esophageal irritation. This method is also useful for improving patient acceptance and compliance.

### Example 15

Once-Weekly Dosing Regimen for Treatment of Osteoarthritis

The alendronate formulation from Example 7 containing about 280 mg of alendronate in about 75 mL of liquid, on an alendronic acid active basis, is orally administered to a human patient once-weekly, such as, about once every seven days (for example, every Sunday), for a period of at least one to six months. This method of administration is useful and convenient for treating osteoarthritis, particularly in humans with difficulty in swallowing tablets, while minimizing adverse upper gastrointestinal effects, particularly esophageal irritation. This method is also useful for improving patient acceptance and compliance.

### Example 16

Once-Weekly Dosing Regimen for Treatment of Metastatic Bone Disease

The alendronate formulation from Example 7 containing about 280 mg of alendronate in about 75 mL of liquid, on an alendronic acid active basis, is orally administered to a human patient once-weekly, such as, about once every seven days (for example, every Sunday). This method of administration is useful and convenient for treating metastatic bone disease in humans with lung, breast, and prostate cancer. The formulation is particularly beneficial in lung, breast, or prostate cancer patients who experience difficulty in swallowing tablets, while minimizing adverse upper gastrointestinal effects, particularly esophageal irritation. This method is also useful for improving patient acceptance and compliance.

### Example 17

Once-Monthly Dosing Regimen for Prevention of Metastatic Bone Disease

The alendronate formulation from Example 7 containing about 280 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating or preventing metastatic bone disease in humans with lung, breast, and prostate cancer. The formulation is particularly beneficial in lung, breast, or prostate cancer patients who experience difficulty in swallowing tablets, while minimizing adverse upper gastrointestinal effects, particularly esophageal irritation. This method is also useful for improving patient acceptance and compliance.

### Example 18

Once-Monthly Dosing Regimen for Prevention of Osteoporosis

The alendronate formulation from Example 7 containing about 280 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for the prevention of osteoporosis, particularly in humans with difficulty in swallowing tablets, while minimizing adverse upper gastrointestinal effects, particularly esophageal irritation. This method is also useful for improving patient acceptance and compliance.

### Example 19

Once-Weekly Dosing Regimen for Treatment of Hypercalcemia of Malignancy

The alendronate formulation from Example 7 containing about 280 mg of alendronate in about 75 mL of liquid, on an alendronic acid active basis, is orally administered to a human patient once-weekly, such as, about once every seven days (for example, every Sunday), for a period of at least one to six months. This method of administration is useful and convenient for treating hypercalcemia of malignancy, particularly in humans with difficulty in swallowing tablets, while minimizing adverse upper gastrointestinal effects, particularly esophageal irritation. This method is also useful for improving patient acceptance and compliance.

### Example 20

Once-Weekly Dosing Regimen for Treatment of Periodontal Disease

The alendronate formulation from Example 7 containing about 280 mg of alendronate in about 75 mL of liquid, on an alendronic acid active basis, is orally administered to a human patient once-weekly, such as, about once every seven days (for example, every Sunday), for a period of at least one to six months. This method of administration is useful and convenient for treating periodontal, particularly in humans with difficulty in swallowing tablets, while minimizing adverse upper gastrointestinal effects, particularly esophageal irritation. This method is also useful for improving patient acceptance and compliance.

### Example 21

Once-Monthly Dosing Regimen for Prevention of Periodontal Disease

The alendronate formulation from Example 7 containing about 280 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for the prevention of periodontal disease, particularly in humans with difficulty in swallowing tablets, while minimizing adverse upper gastrointestinal effects, particularly esophageal irritation. This method is also useful for improving patient acceptance and compliance.

### Example 22

Once-Weekly Dosing Regimen for Treatment of Periprosthetic Osteolysis

The alendronate formulation from Example 7 containing about 280 mg of alendronate in about 75 mL of liquid, on an alendronic acid active basis, is orally administered to a human patient once-weekly, such as, about once every seven days (for example, every Sunday), for a period of at least one to six months. This method of administration is useful and convenient for treating, periprosthetic osteolysis particularly in humans with difficulty in swallowing tablets, while minimizing adverse upper gastrointestinal effects, particularly esophageal irritation. This method is also useful for improving patient acceptance and compliance.

### Example 23

A liquid formulation containing 375 mg of alendronate monosodium trihydrate, on an alendronic acid active basis, in about 75 mL of liquid was prepared using the following relative weights of ingredients:

| | |
|---|---|
| Alendronate monosodium trihydrate* | 6.527 mg/mL |
| Sodium Citrate | 21.18 mg/mL |
| Sodium hydroxide | qs pH 6.8 |
| Hydrochloric acid | qs pH 6.8 |
| Purified Water | qs 1.00 mL |

*Corresponds to 5.0 mg free alendronic acid equivalents or 375 mg/75 mL

Additional agents such as cosolvents, flavoring agents, coloring agents, preservatives, and stabilizers can also be specifically incorporated in the formulation as follows:

| | |
|---|---|
| Alendronate monosodium trihydrate* | 6.527 mg/mL |
| Sodium Citrate | 21.18 mg/mL |
| Sodium propylparaben | 0.2250 mg/mL |
| Sodium butylparaben | 0.07500 mg/mL |
| Sodium saccharin | 0.100 mg/mL |
| Flavor | qs for taste |
| Sodium hydroxide | qs pH 6.8 |
| Hydrochloric acid | qs pH 6.8 |
| Purified Water | qs 1.00 mL |

*Corresponds to 5.0 mg free alendronic acid equivalents or 375 mg/75 mL

The method of manufacture was the same as that for Example 1.

### Example 24

Once-Monthly Dosing Regimen for Treatment of Paget's Disease

The alendronate formulation from Example 23 containing about 375 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating Paget's disease, particularly in humans with difficulty in swallowing tablets, while minimizing adverse upper gastrointestinal effects, particularly esophageal irritation. This method is also useful for improving patient acceptance and compliance.

### Example 25

A liquid formulation containing 560 mg of alendronate monosodium trihydrate, on an alendronic acid active basis, in about 75 mL of liquid was prepared using the following relative weights of ingredients:

| | |
|---|---|
| Alendronate monosodium trihydrate* | 9.748 mg/mL |
| Sodium Citrate | 21.18 mg/mL |
| Sodium hydroxide | qs pH 6.8 |
| Hydrochloric acid | qs pH 6.8 |
| Purified Water | qs 1.00 mL |

*Corresponds to 7.467 mg free alendronic acid equivalents or 560 mg/75 mL

Additional agents such as cosolvents, flavoring agents, coloring agents, preservatives, and stabilizers can also be specifically incorporated in the formulation as follows:

| | |
|---|---|
| Alendronate monosodium trihydrate* | 9.748 mg/mL |
| Sodium Citrate | 21.18 mg/mL |
| Sodium propylparaben | 0.2250 mg/mL |
| Sodium butylparaben | 0.07500 mg/mL |
| Sodium saccharin | 0.100 mg/mL |
| Flavor | qs for taste |
| Sodium hydroxide | qs pH 6.8 |
| Hydrochloric acid | qs pH 6.8 |
| Purified Water | qs 1.00 mL |

*Corresponds to 7.467 mg free alendronic acid equivalents or 560 mg/75 mL

The method of manufacture was the same as that for Example 1.

### Example 26

Biweekly Dosing Regimen for Treatment of Paget's Disease of Bone

The alendronate formulation from Example 25 containing about 560 mg of alendronate in about 75 mL of liquid, on an alendronic acid active basis, is orally administered to a human patient biweekly, such as, about once every fourteen days (for example, on alternate Sundays), for a period of at least one to six months. This method of administration is useful and convenient for treating Paget's disease of bone, particularly in humans with difficulty in swallowing tablets, while minimizing adverse upper gastrointestinal effects, particularly esophageal irritation. This method is also useful for improving patient acceptance and compliance.

### Example 27

Once-Monthly Dosing Regimen for Treatment of Paget's Disease of Bone

The alendronate formulation from Example 25 containing about 560 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating Paget's disease, particularly in humans with difficulty in swallowing tablets, while minimizing adverse upper gastrointestinal effects, particularly esophageal irritation. This method is also useful for improving patient acceptance and compliance.

### Example 28

Once-Weekly Dosing Regimen for Treatment of Paget's Disease of Bone

The alendronate formulation from Example 25 containing about 560 mg of alendronate in about 75 mL of liquid, on an alendronic acid active basis, is orally administered to a human patient once-weekly, such as, about once every seven days (for example, every Sunday), for a period of at least one to six months. This method of administration is useful and convenient for treating Paget's disease of bone, particularly in humans with difficulty in swallowing tablets, while minimizing adverse upper gastrointestinal effects, particularly esophageal irritation. This method is also useful for improving patient acceptance and compliance.

### Example 29

Once-Monthly Dosing Regimen for Treatment of Osteoporosis

The alendronate formulation from Example 25 containing about 560 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating osteoporosis, particularly in humans with difficulty in swallowing tablets, while minimizing adverse upper gastrointestinal effects, particularly esophageal irritation. This method is also useful for improving patient acceptance and compliance.

### Example 30

Biweekly Dosing Regimen for Treatment of Osteoarthritis

The alendronate formulation from Example 25 containing about 560 mg of alendronate in about 75 mL of liquid, on an alendronic acid active basis, is orally administered to a human patient biweekly, such as, about once every fourteen days (for example, on alternate Sundays), for a period of at least one to six months. This method of administration is useful and convenient for treating osteoarthritis, particularly in humans with difficulty in swallowing tablets, while minimizing adverse upper gastrointestinal effects, particularly esophageal irritation. This method is also useful for improving patient acceptance and compliance.

### Example 31

Once-Monthly Dosing Regimen for Treatment of Osteoarthritis

The alendronate formulation from Example 25 containing about 560 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating osteoarthritis, particularly in humans with difficulty in swallowing tablets, while minimizing adverse upper gastrointestinal effects, particularly esophageal irritation. This method is also useful for improving patient acceptance and compliance.

### Example 32

Biweekly Dosing Regimen for Treatment or Prevention of Metastatic Bone Disease

The alendronate formulation from Example 25 containing about 560 mg of alendronate in about 75 mL of liquid, on an alendronic acid active basis, is orally administered to a human patient biweekly, such as, about once every fourteen days (for example, on alternate Sundays), for a period of at least one to six months. This method of administration is useful and convenient for treating or preventing metastatic bone disease in humans with lung, breast, and prostate cancer. The formulation is particularly beneficial in lung, breast, or prostate cancer patients who experience difficulty in swallowing tablets, while minimizing adverse upper gastrointestinal effects, particularly esophageal irritation.

### Example 33

Once-Monthly Dosing Regimen for Treatment or Prevention of Metastatic Bone Disease

The alendronate formulation from Example 25 containing about 560 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating or preventing metastatic bone disease in humans with lung, breast, and prostate cancer. The formulation is particularly beneficial in lung, breast, or prostate cancer patients who experience difficulty in swallowing tablets, while minimizing adverse upper gastrointestinal effects, particularly esophageal irritation.

### Example 34

A liquid formulation containing about 1120 mg of alendronate monosodium trihydrate, on an alendronic acid active basis, in about 75 mL of liquid is prepared using the following relative weights of ingredients:

| | |
|---|---|
| Alendronate monosodium trihydrate* | 19.495 mg/mL |
| Sodium Citrate | 21.18 mg/mL |
| Sodium hydroxide | qs pH 6.8 |
| Hydrochloric acid | qs pH 6.8 |
| Purified Water | qs 1.00 mL |

*Corresponds to 14.93 mg free alendronic acid equivalents or 1120 mg/75 mL

Additional agents such as cosolvents, flavoring agents, coloring agents, preservatives, and stabilizers can also be specifically incorporated in the formulation as follows:

| | |
|---|---|
| Alendronate monosodium trihydrate* | 19.495 mg/mL |
| Sodium Citrate | 21.18 mg/mL |
| Sodium propylparaben | 0.2250 mg/mL |
| Sodium butylparaben | 0.07500 mg/mL |
| Sodium saccharin | 0.100 mg/mL |
| Flavor | qs for taste |
| Sodium hydroxide | qs pH 6.8 |
| Hydrochloric acid | qs pH 6.8 |
| Purified Water | qs 1.00 mL |

*Corresponds to 14.93 mg free alendronic acid equivalents or 1120 mg/75 mL

The method of manufacture is the same as that for Example 1.

### Example 35

Biweekly Dosing Regimen for Treatment of Paget's Disease of Bone

The alendronate formulation from Example 34 containing about 1120 mg of alendronate in about 75 mL of liquid, on an alendronic acid active basis, is orally administered to a human patient biweekly, such as, about once every fourteen days (for example, on alternate Sundays), for a period of at least one to six months. This method of administration is useful and convenient for treating Paget's disease of bone, particularly in humans with difficulty in swallowing tablets, while minimizing adverse upper gastrointestinal effects, particularly esophageal irritation. This method is also useful for improving patient acceptance and compliance.

### Example 36

Once-Monthly Dosing Regimen for Treatment of Paget's Disease of Bone

The alendronate formulation from Example 34 containing about 1120 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating Paget's disease, particularly in humans with difficulty in swallowing tablets, while minimizing adverse upper gastrointestinal effects, particularly esophageal irritation. This method is also useful for improving patient acceptance and compliance.

### Example 37

Once-Monthly Dosing Regimen for Treatment of Metastatic Bone Disease

The alendronate formulation from Example 34 containing about 1120 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating metastatic bone disease in humans with lung, breast, and prostate cancer. The formulation is particularly beneficial in lung, breast, or prostate cancer patients who experience difficulty in swallowing tablets, while minimizing adverse upper gastrointestinal effects, particularly esophageal irritation.

### Example 38

Once-Monthly Dosing Regimen for Treatment of Osteoarthritis

The alendronate formulation from Example 34 containing about 1120 mg of alendronate in about 75 mL of liquid, on an alendronic acid active basis, is orally administered to a human patient once-monthly, such as, about once every 28, 30, or 31 days (for example, the 1^{st} of every month), for a period of at least six months to a year and possibly for numerous consecutive years. This method of administration is useful and convenient for treating osteoarthritis, particularly in humans with difficulty in swallowing tablets, while minimizing adverse upper gastrointestinal effects, particularly esophageal irritation. This method is also useful for improving patient acceptance and compliance.

What is claimed is:

1. A method for treating or preventing a bone disorder in a mammal in need thereof comprising orally administering to said mammal a pharmaceutically effective amount of a liquid pharmaceutical composition of at least one bisphosphonate, or a pharmaceutically acceptable salt thereof, according to a dosing schedule having a dosing interval selected from once-weekly dosing, twice-weekly dosing, biweekly dosing, twice-monthly dosing, and once-monthly dosing.

2. A method for treating or preventing a bone disorder in a mammal in need thereof comprising orally administering to said mammal a pharmaceutically effective amount of a liquid pharmaceutical composition of at least one bisphosphonate, or a pharmaceutically acceptable salt thereof, as a unit dosage which ranges from greater than about 140 mg to about 1120 mg, on a bisphosphonic acid active basis, according to a dosing schedule having a dosing interval selected from once-weekly dosing, twice-weekly dosing, biweekly dosing, twice-monthly dosing, and once-monthly dosing.

3. The method of claim 2, wherein said at least one bisphosphonate is selected from alendronate, clodronate, etidronate, ibandronate, incadronate, minodronate, neridronate, olpadronate, pamidronate, piridronate, risedronate, tiludronate, zoledronate, and pharmaceutically acceptable salts thereof.

4. The method of claim 3, wherein said bone disorder is selected from Paget's disease, osteoporosis, metastatic bone disease, hypercalcemia of malignancy, periprosthetic osteolysis, periodontal disease and arthritic conditions.

5. The method of claim 4 wherein said bone disorder is Paget's disease.

6. The method of claim 5, wherein said at least one bisphosphonate is selected from alendronate, pharmaceutically acceptable salts thereof, and mixtures thereof.

7. The method of claim 4, wherein said liquid pharmaceutical composition is an aqueous solution.

8. The method of claim 7, wherein said dosing interval is once-weekly.

9. The method of claim 8 wherein said unit dosage of alendronate ranges from about 280 mg to about 560 mg, on an alendronic acid active basis.

10. The method of claim 7, wherein said dosing interval is twice-weekly.

11. The method of claim 10 wherein said unit dosage of alendronate ranges from greater than about 140 mg to about 280 mg, on an alendronic acid active basis.

12. The method of claim 7, wherein said dosing interval is biweekly.

13. The method of claim 12 wherein said unit dosage of alendronate ranges from about 280 mg to about 1120 mg, on an alendronic acid active basis.

14. The method of claim 7, wherein said dosing interval is once monthly.

15. The method of claim 14 wherein said unit dosage of alendronate ranges from about 280 mg to about 1120 mg, on an alendronic acid active basis.

16. The method of claim 4, wherein said bone disorder is osteoporosis.

17. The method of claim 16, wherein said at least one bisphosphonate is selected from alendronate, pharmaceutically acceptable salts thereof, and mixtures thereof.

18. The method of claim 16, wherein said liquid pharmaceutical composition is an aqueous solution.

19. The method of claim 18, wherein said dosing interval is once-weekly.

20. The method of claim 19 wherein said unit dosage of alendronate is about 280 mg, on an alendronic acid active basis.

21. The method of claim 18, wherein said dosing interval is twice-weekly.

22. The method of claim 21 wherein said unit dosage of alendronate ranges from greater than about 140 mg to about 280 mg, on an alendronic acid active basis.

23. The method of claim 18, wherein said dosing interval is biweekly.

24. The method of claim 23 wherein said unit dosage of alendronate is about 280 mg, on an alendronic acid active basis.

25. The method of claim 18, wherein said dosing interval is once monthly.

26. The method of claim 25 wherein said unit dosage of alendronate ranges from about 280 mg to about 1120 mg, on an alendronic acid active basis.

27. The method of claim 4, wherein said bone disorder is metastatic bone disease.

28. The method of claim 5, wherein said at least one bisphosphonate is selected from alendronate, pharmaceutically acceptable salts thereof, and mixtures thereof.

29. The method of claim 27, wherein said liquid pharmaceutical composition is an aqueous solution.

30. The method of claim 29, wherein said dosing interval is once-weekly.

31. The method of claim 30 wherein said unit dosage of alendronate is about 280 mg, on an alendronic acid active basis.

32. The method of claim 29, wherein said dosing interval is twice-weekly.

33. The method of claim 32 wherein said unit dosage of alendronate ranges from greater than about 140 mg to about 280 mg, on an alendronic acid active basis.

34. The method of claim 29, wherein said dosing interval is biweekly.

35. The method of claim 34 wherein said unit dosage of alendronate is about 560 mg, on an alendronic acid active basis.

36. The method of claim 29, wherein said dosing interval is once monthly.

37. The method of claim 36 wherein said unit dosage of alendronate ranges from about 560 mg to about 1120 mg, on an alendronic acid active basis.

38. The method of claim 4, wherein said bone disease is osteoarthritis.

39. The method of claim 38, wherein said at least one bisphosphonate is selected from alendronate, pharmaceutically acceptable salts thereof, and mixtures thereof.

40. The method of claim 38, wherein said liquid pharmaceutical composition is an aqueous solution.

41. The method of claim 40, wherein said dosing interval is once-weekly.

42. The method of claim 41 wherein said unit dosage of alendronate is about 280 mg, on an alendronic acid active basis.

43. The method of claim 40, wherein said dosing interval is twice-weekly.

44. The method of claim 43 wherein said unit dosage of alendronate is greater than about 140 mg on an alendronic acid active basis.

45. The method of claim 40, wherein said dosing interval is biweekly.

46. The method of claim 45 wherein said unit dosage of alendronate is about 560 mg, on an alendronic acid active basis.

47. The method of claim 40, wherein said dosing interval is once monthly.

48. The method of claim 47 wherein said unit dosage of alendronate ranges from about 560 mg to about 1120 mg, on an alendronic acid active basis.

49. An oral liquid pharmaceutical composition comprising:
a) a therapeutically effective amount of at least one bisphosphonate or a pharmaceutically acceptable salt thereof,
b) a pharmaceutically acceptable carrier, and
c) a pharmaceutically acceptable buffer,
wherein a dose of said oral liquid pharmaceutical composition has a buffering capacity sufficient to buffer at least 50 mL of 0.1 N HCl to a pH of greater than or equal to 3.5.

50. An oral liquid pharmaceutical composition according to claim 49, wherein said at least one bisphosphonate or pharmaceutically acceptable salt is present in a dose amount ranging from greater than about 140 mg to about 1335 mg on an alendronic acid active weight basis.

51. An oral liquid pharmaceutical composition according to claim 50, wherein said at least one bisphosphonate is alendronic acid or a pharmaceutically acceptable salt of alendronic acid.

52. An oral liquid pharmaceutical composition according to claim 51, wherein said pharmaceutically acceptable salt of alendronic acid is a monosodium salt.

53. An oral liquid pharmaceutical composition according to claim 49, wherein said pharmaceutically acceptable buffer is selected from citrate, tartrate, phosphate, fumarate, succinate, and acetate.

54. An oral liquid pharmaceutical composition according to claim 53, wherein said pharmaceutically acceptable buffer is citrate.

55. An oral liquid pharmaceutical composition according to claim 54, wherein the oral liquid pharmaceutical composition has a pH ranging from about 5 to about 10.

56. An oral liquid pharmaceutical composition according to claim 55, wherein the oral liquid pharmaceutical composition has a pH ranging from about 6.4 to about 7.2.

57. An oral liquid pharmaceutical composition according to claim 51, wherein the concentration of said alendronic acid or a pharmaceutically acceptable salt of alendronic acid ranges from about 1.86 mg/mL to about 17.8 mg/mL on an alendronic acid active weight basis.

58. An oral liquid pharmaceutical composition according to claim 57, wherein the concentration of said alendronic acid or a pharmaceutically acceptable salt of alendronic acid ranges from about 2.5 mg/mL to about 14.93 mg/mL on an alendronic acid active weight basis.

59. An oral liquid pharmaceutical composition according to claim 58, wherein the concentration of said alendronic acid or a pharmaceutically acceptable salt of alendronic acid ranges from about 3.0 mg/mL to about 7.5 mg/mL on an alendronic acid active weight basis.

60. An oral liquid pharmaceutical composition according to claim 49, wherein the dose of said oral liquid pharmaceutical composition has a volume ranging from about 30 mL to about 150 mL.

61. An oral liquid pharmaceutical composition according to claim 60, wherein the dose of said oral liquid pharmaceutical composition has a volume ranging from about 75 mL to about 100 mL.

62. An oral liquid pharmaceutical composition according to claim 49, further comprising a preservative system.

63. An oral liquid pharmaceutical composition according to claim 62, wherein said preservative system comprises at least one compound selected from ethanol, parabens, benzoic acid, sorbic acid, and pharmaceutically acceptable salts, thereof.

64. An oral liquid pharmaceutical composition according to claim 63, wherein said parabens and pharmaceutically acceptable salts of said parabens are selected from ethylparaben, methylparaben, propylparaben, sodium propylparaben, and sodium butylparaben.

65. An oral liquid pharmaceutical composition according to claim 49, further comprising at least one pharmaceutically acceptable sweetener.

66. An oral liquid pharmaceutical composition according to claim 65, wherein said pharmaceutically acceptable sweetener is selected from saccharin, pharmaceutically acceptable salts of saccharin, aspartame acesulfame-K, sucrose, dextrose, maltose, fructose, galactose, dextrose and glycerin.

67. An oral liquid pharmaceutical composition according to claim 49, further comprising at least one agent selected from flavors and colors.

68. An oral liquid pharmaceutical composition of greater than about 140 mg to about 1335 mg of alendronate on an alendronic acid active weight basis having the following formula:

| Ingredient | Concentration |
|---|---|
| Alendronate monosodium | 2.43-23.24 mg/mL |
| trihydrate | |
| Citrate | 72 mM |
| Sodium propylparaben | 0.12-0.5 mg/mL |
| Sodium butylparaben | 0.04-0.2 mg/mL |
| Sodium saccharin | 0-5 mg/mL |
| NaOH | pH adjustment (pH 3- |
| | 10) |
| HCl | pH adjustment (pH 3- |
| | 10) |
| Water | Qs 1 mL |

69. An oral liquid pharmaceutical composition of about 280 mg of alendronate on an alendronic acid active weight basis having the following formula:

| Ingredient | Concentration |
|---|---|
| Alendronate monosodium | 4.87 mg/mL |
| trihydrate | |
| Sodium Citrate dihydrate | 21.2 mg/mL |
| Sodium propylparaben | 0.2 mg/mL |
| Sodium butylparaben | 0.08 mg/mL |
| Sodium saccharin | 0.1 mg/mL |
| NaOH | pH adjustment |
| | (pH 6.4-7.2) |
| HCl | pH adjustment |
| | (pH 6.4-7.2) |
| Water | Qs 1 mL |

70. A process for making an oral liquid pharmaceutical composition comprising combining:
a) a therapeutically effective amount of at least one bisphosphonate or a pharmaceutically acceptable salt thereof,
b) a pharmaceutically acceptable carrier, and
c) a pharmaceutically acceptable buffer,
wherein a dose of said oral liquid pharmaceutical composition has a buffering capacity sufficient to buffer at least 50 mL of 0.1 N HCl to a pH of greater than or equal to 3.5.

71. A process according to claim 70, wherein said at least one bisphosphonate is present in said oral liquid pharmaceutical composition in amount ranging from greater than about 140 mg to about 1335 mg on an alendronic acid active weight basis.

72. A process according to claim 71, wherein said at least one bisphosphonate is chosen from alendronic acid, a salt of alendronic acid and mixtures thereof.

73. A process according to claim 72, wherein said salt of alendronic acid is chosen from a sodium salt, a potassium salt, a calcium salt, a magnesium salt, an ammonium salt, and mixtures thereof.

74. A process according to claim 71, wherein said at least one bisphosphonate is chosen from the monosodium trihydrate salt of alendronic acid and the monosodium monohydrate salt of alendronic acid.

75. An oral liquid pharmaceutical composition made by the process of claim 71.

76. An oral liquid pharmaceutical composition made by the process of claim 74.

77. An oral liquid pharmaceutical composition according to claim 49, wherein said dose of said oral liquid pharmaceutical composition has a buffering capacity sufficient to buffer 100 mL of 0.1 N HCl to a pH of greater than or equal to 3.5.

78. An oral liquid pharmaceutical composition according to claim 49, wherein said dose of said oral liquid pharmaceutical composition has a buffering capacity sufficient to buffer 75 mL of 0.1 N HCl to a pH of greater than or equal to 3.5.

79. An oral liquid pharmaceutical composition according to claim 49, further comprising a supplemental amount of vitamin D.

80. A method to treat or prevent osteoporosis comprising the once-monthly dosing of greater than about 140 to about 280 mg of said liquid pharmaceutical composition of claim 49.

81. A method to treat osteoporosis comprising the once-weekly dosing of about 280 mg of said liquid pharmaceutical composition of claim 49.

82. A method to prevent osteoporosis comprising the once-monthly dosing of greater than about 140 mg of said liquid pharmaceutical composition of claim 49.

83. A method to treat Paget's disease comprising the once-weekly dosing of about 280 mg of said liquid pharmaceutical composition of claim 49.

84. A method to prevent metastatic bone disease comprising the once-monthly dosing of about 280 mg of said liquid pharmaceutical composition of claim 49.

85. A method to treat metastatic bone disease comprising the once-weekly dosing of about 280 mg of said liquid pharmaceutical composition of claim 49.

86. A method to treat osteoarthritis comprising the once-weekly dosing of about 280 mg of said liquid pharmaceutical composition of claim 49.

87. A method to prevent osteoarthritis comprising the once-monthly dosing of about 280 mg of said liquid pharmaceutical composition of claim 49.

88. A method to treat hypercalcemia of malignancy comprising the once-weekly dosing of about 280 mg of said liquid pharmaceutical composition of claim 49.

89. A method to treat periodontal disease comprising the once-weekly dosing of about 280 mg of said liquid pharmaceutical composition of claim 49.

90. A method to prevent periodontal disease comprising the once-monthly dosing of about 280 mg of said liquid pharmaceutical composition of claim 49.

91. A method to treat periprosthetic osteolysis comprising the once-weekly dosing of about 280 mg of said liquid pharmaceutical composition of claim 49.
